(19) United States
(12) Patent Application Publication
(10) Pub. No.: US 2018/0014838 A1
(43) Pub. Date: Jan. 18, 2018

(54) MAGNETIC IMPLANT FOR JOINT FIXATION AND METHOD

(71) Applicant: Omega Innovative Technologies, LLC, Palm Beach Gardens, FL (US)

(72) Inventor: Autumn Ning, Miami, FL (US)

(21) Appl. No.: 15/637,393

(22) Filed: Jun. 29, 2017

Related U.S. Application Data
(60) Provisional application No. 62/356,296, filed on Jun. 29, 2016.

(51) Int. Cl. A61B 17/17; A61B 17/16; A61B 17/52; A61B 17/00; A61B 17/88; A61B 17/70; A61B 17/90; A61B 17/02; A61B 17/12; A61B 17/68

(52) U.S. Cl. CPC A61B 17/1707; A61B 17/88; A61B 17/1671; A61B 17/52; A61B 17/7032; A61B 17/7038; A61B 17/8872; A61B 17/00234; A61B 17/7089; A61B 2017/00876; A61B 2017/0275; A61B 2017/12086; A61B 2017/681; A61B 2017/90

(57) ABSTRACT

The present invention provides for a joint fixation device and method utilizing magnetic members with at least one magnetic member positioned on each of opposite sides of a joint and secured to skeletal components. The magnetic members include at least one permanent magnet. The magnetic members can be configured to provide an attracting force or a repelling force. They can also be configured to provide a torque about the joint.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 62/356,296, entitled “MAGNETIC IMPLANT FOR JOINT FIXATION”, filed Jun. 29, 2016. The contents of which the above referenced application is incorporated herein by reference.

### FIELD OF THE INVENTION

The instant invention relates to a magnetic implant for skeletal joint fixation procedures and methods of use thereof; and more particularly, to a pedicle screw magnet for spinal joint alignment fixation and a magnetic insert for joint, such as a hip joint, fixation.

### BACKGROUND OF THE INVENTION

Joints in the human body often need the bones on opposite sides thereof positionally fixed relative to one another. Such fixation can be needed to correct spinal alignment and to hold replacement joints, such as hip, shoulder and elbow joints together.

In one embodiment, the central nervous system is a vital part of the human physiology that coordinates human activity. It is primarily made up of the brain and the spinal cord. The spinal cord is made up of a bundle of nerve tissue which originates in the brain and branches out to various parts of the body, acting as a conduit to communicate neuronal signals from the brain to the rest of the body, including motor control and sensations. Protecting the spinal cord is the spinal, or vertebral, column. Anatomically, the spinal column is made up of several regions, including the cervical, thoracic, lumbar and sacral regions. The cervical spine is made up of seven vertebrae and functions to support the weight of the head. The thoracic spine is made up of twelve vertebrae and functions to protect the organs located within the chest. Five vertebrae make up the lumbar spine. The lumbar spine contains the largest vertebra and functions as the main weight bearing portion of the spine. Located at the base of the spine are the five fused vertebrae known as the sacrum. The coccyx sits at the base of the spinal column and consists of four fused vertebrae.

Each of the vertebrae associated with the various spinal cord regions are made up of a vertebral body, a posterior arch, and transverse processes. The vertebral body, often described as having a drum-like shape, is designed to bear weight and withstand compression or loading. In between the vertebral bodies is a joint containing an intervertebral disc forming part of a vertebral joint. The intervertebral disc is filled with a soft, gelatinous-like substance which helps cushion the spine against various movements and can be the source of various diseases. The posterior arch of the vertebrae is made up of the lamina, pedicles and facet joints. Transverse processes extend outwardly from the vertebrae and provide the means for muscle and ligament attachment, which aid in movement and stabilization of the vertebrae.

While most people have fully functional spinal cords, it is not uncommon for individuals to suffer some type of spinal ailment, including spondylolisthesis, scoliosis, or spinal fractures. One of the more common disorders associated with the spinal cord is damage to the spinal discs. Damage to the discs results from physical injury, disease, genetic disposition, or as part of the natural aging process. Disc damage often results in intervertebral spacing not being maintained, causing pinching of exiting nerve roots between the discs, resulting in pain. For example, disc herniation is a condition in which the disc substance bulges from the disc space between the two vertebrae bodies. It is the bulging of the disc material which causes impingement on the nerves, manifesting in pain to the patient. For most patients, rest and administration of pain and anti-inflammatory medications alleviates the problem. However, in severe cases, cases which have developed into spinal instability or severe disc degeneration, the damaged disc material between the vertebral bodies is removed and replaced with spinal stabilization implants. Restoration to the normal height allows the pressure on the nerve roots to be relieved.

There are many different approaches taken to alleviate or reduce severe spinal disorders. One surgical procedure commonly used is a spinal fusion technique. Several surgical approaches have been developed over the years, and include the Posterior Lumbar Interbody Fusion (PLIF) procedure which utilizes a posterior approach to access the patient's vertebrae or disc space, the Transforaminal Lumbar Interbody Fusion (TLIF) procedure which utilizes a posterior and lateral approach to access the patient's vertebrae or disc space, and the Anterior Lumbar Interbody Fusion (ALIF) which utilizes an anterior approach to access the patient's vertebrae or disc space. Using any of these surgical procedures, the patient undergoes spinal fusion surgery in which two or more vertebrae are linked or fused together through the use of a bone spacing device and/or use of bone grafts. The resulting surgery eliminates any movement between the spinal sections which have been fused together.

In addition to the spinal implants or use of bone grafts, spinal fusion surgery often utilizes spinal instrumentation or surgical hardware, such as pedicle screws, plates, or spinal rods. Once the spinal spacers and/or bone grafts have been inserted, a surgeon places the pedicle screws into a portion of the spinal vertebrae and attaches either rods or plates to the screws as a means for stabilization while the bones fuse. Currently, available systems for inserting the rods into pedicle screws can be difficult, particularly in light of the fact that surgeons installing these rods often work in narrow surgical fields. Moreover, since patients can vary with respect to their internal anatomy, resulting in varying curvatures of the spine, a surgeon may not always have a linear path or may have anatomical structures that must be maneuvered around in order to properly insert the surgical rods into the pedicle screw assemblies. In addition to requiring surgical skill, difficulty in placing the rods correctly into the pedicle screws can result in unnecessary increases in the time it takes a surgeon to complete the surgical procedure. Prolonged surgery times increase the risk to the patient. More importantly, improperly aligning the rods and pedicle screw assemblies often results in complications for the patient and requires corrective surgical procedures.

Joints are now commonly replaced with manmade joints. One joint commonly replaced is the hip joint, wherein the ball and socket are replaced. The joint is normally fixed together by the muscles that held the original joint together, which in some cases, may not be adequate fixation.

Muscles play an important role in the functioning and integrity of these joints. In the case of the spine, muscles help keep the vertebrae aligned and, in the case of a ball and socket joint, keep the ball in the socket. However, help is often needed in supplementing muscle action in fixing joint components together due to deformity, age or accidental injury.

There exists, therefore, a need for an improved bone joint fixation means to assist in keeping joint components properly fixed relative to one another.

### DESCRIPTION OF THE PRIOR ART

U.S. Pat. No. 6,530,929 discloses an installation instrument for placement of a brace or rod into pedicle screws. The instrument is mounted to anchors secured to the pedicle screws utilizing extensions coupled to the anchors. The instrument is movable with respect to the anchors to position a brace in a position more proximate the anchors. The brace can be inserted into the pedicle screws and manipulated away from the installation instrument utilizing a thumb screw. However, a disadvantage associated with the installation instrument for placement of a brace or rod into pedicle screws described therein is that the brace cannot be rotated about its longitudinal axis.

U.S. Pat. No. 7,188,626 discloses methods and instruments for placing a brace or connecting element into a plurality of anchors or pedicle screws similar to U.S. Pat. No. 6,530,929. Insertion of the connecting elements is accomplished by a linear insertion method, therefore failing to teach a connecting element that can to be rotated about its longitudinal axis.

U.S. Pat. No. 7,520,879 discloses a device for positioning a connecting element adjacent the spinal column using minimally invasive procedures. An inserter instrument guides the connecting element from a location remote from one or more anchors to a location proximate to the one or more anchors. The extensions are mountable to anchors, and the inserter instrument is mountable to the connecting element for positioning the connecting element adjacent the anchors in a minimally invasive procedure. The inserter instrument does not have to be mounted to the anchors or to the anchor extensions, and is operable independently to position the connecting element into the patient along a minimally invasive insertion path from a location remote from the anchor extensions. While the inserter instrument can rotate the connecting element along its longitudinal axis, it cannot be repositioned on the connecting element to gradually rotate the connecting element in a given direction. Moreover, it cannot be rotated about an axis normal to its longitudinal axis.

U.S. Publication No. 2007/0078460 discloses a method and instrumentation for performing spinal fixation surgery. A first incision is made through the skin, and a passageway is created to the spine. A screw is inserted through the passageway and into a vertebra. The screw has a head portion including a channel. An insertion guide is operably connected to the screw. Additional screws may each be inserted through separate incisions or through the first incision. Insertion guides may be operably connected to a head portion of each screw. A sleeve may be positioned into one insertion guide in a first position to guide a rod through at least one other insertion guide. The sleeve is rotated to a second position to allow the rod to move down the slots of the insertion guides and into the head portion of the screw. Additionally a holding instrument can be employed to position a rod. Two types of connections between the holding instrument and the rod are described. These connections permit the rotation of the rod about its longitudinal axis, but fail to teach a rod which can be repositioned on the connecting element to gradually rotate the connecting element in a given direction.

U.S. Published Application No. 2005/0277934 discloses a minimally invasive spinal fixation system used for spinal arthrodesis or motion preservation spinal repair. The system includes a plurality of pedicle screws, and an attachment assembly for connecting the pedicle screws. The attachment assembly includes a connector for attaching to the first screw and second screw, and a removable guide for percutaneously attaching the connector to the first screw and second screw. The removable guide includes a number of different embodiments for connecting the attachment assembly to the connector. A snap type lock is used to secure the attachment to the connector. While this does permit the connector to be repositioned by rotating it about its longitudinal axis, the repositioning can occur at only 90 degree increments. Moreover, it cannot be rotated about an axis normal to the longitudinal axis of the connector.

Therefore, what is needed is a bone fixation device which does not require a surgeon to use a predetermined, fixed linear pathway when attempting to insert the device into a pedicle screw assembly. A bone fixation device which allows the surgeon the ability to navigate the device while being inserted into a pedicle screw assembly through a non-linear pathway by incrementally changing the direction of travel is also desired.

### SUMMARY OF THE INVENTION

The present invention provides for a bone fixation device that assists in fixing one vertebra in relation to another vertebra on opposite sides of a joint to maintain a proper relationship to one another. The bone fixation device as described herein provides a surgeon with a device that can easily and safely be inserted into a patient to enhance the proper functioning of a joint.

As such, the bone fixation device includes at least two magnetic components that can include at least one magnet implant and another magnetic component fixed to the skeleton on opposite sides of a joint and use at least one magnetic field to continuously apply force to maintain joint component fixation relative to one another. A pair of magnets is preferably utilized. The magnets are preferably permanent magnets.

Accordingly, it is a primary objective of the instant invention to provide an improved bone fixation device which is useful in orthopedic surgeries.

It is a further objective of the instant invention to provide a bone fixation device configured to provide either attracting or repelling force from adjacent magnets.

It is yet another objective of the instant invention to provide a bone fixation device which can be locked in place in order to bias bones on opposite sides of a joint to an angular position relative to one another.

It is yet another objective of the instant invention to provide a bone fixation device which can be configured to bones on opposite sides of a joint to freely move relative to one another.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A is a side elevation sectional view of a pedicle screw;

FIG. 1B is a fragmentary perspective view of the upper portion of a pedicle screw;

FIG. 2 is a front elevation fragmentary view of a spinal cord with magnetic components mounted in place;

FIG. 3 is a schematic view of a pair of magnetic components, illustrating relative position options;

FIG. 4 is a side elevation fragmentary sectional view of magnetic components associated with a ball and socket joint;

FIG. 5 is a perspective view of joint fixation devices secured to vertebrae of a human skeleton; and

FIG. 6 is a perspective view of components of a joint fixation device.

### DETAILED DESCRIPTION OF THE INVENTION

While the present invention is susceptible of embodiment in various forms, there is shown in the drawings and will hereinafter be described presently preferred, albeit not limiting, embodiments with the understanding that the present disclosure is to be considered exemplifications of the present invention and are not intended to limit the invention to the specific embodiments illustrated.

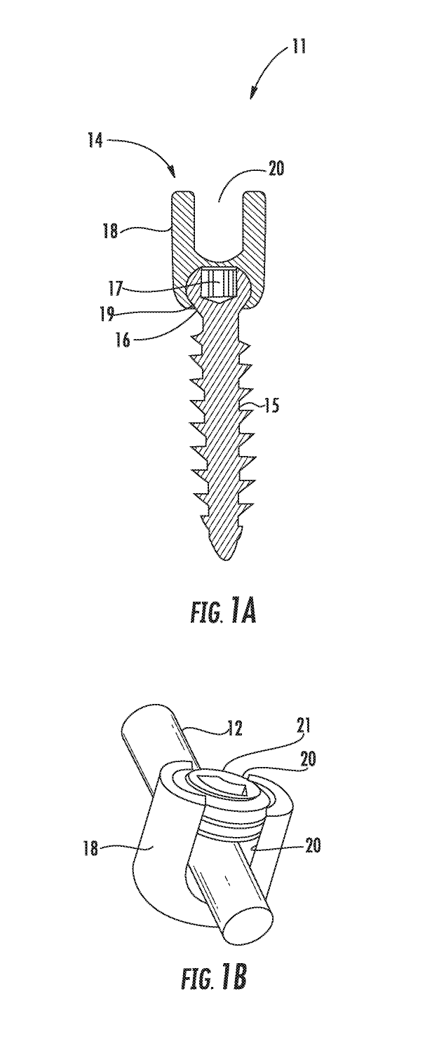

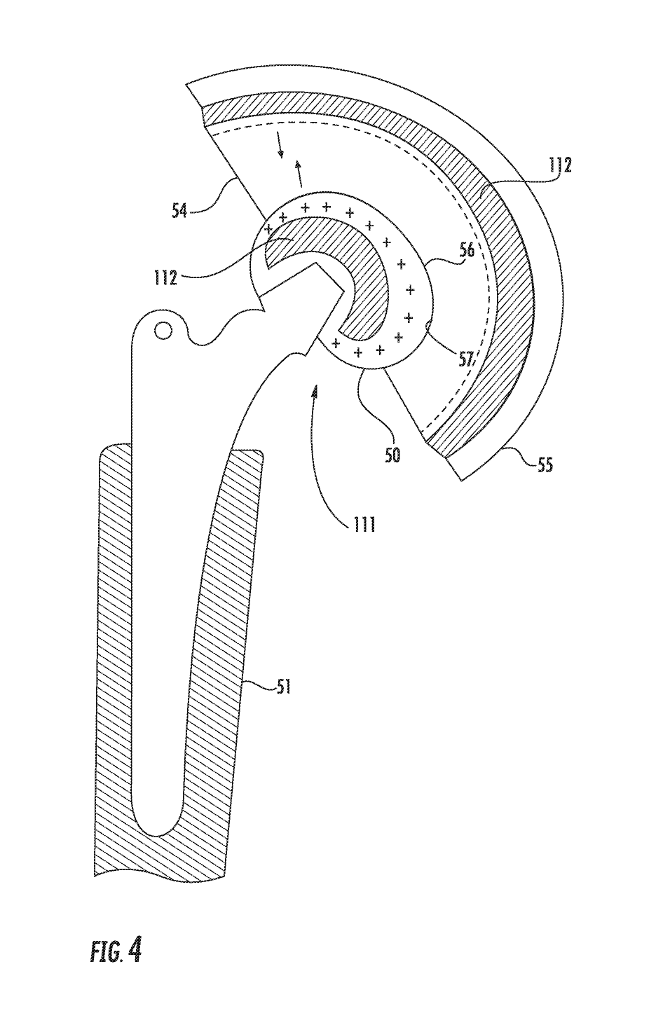

FIGS. 1-3 illustrate a first embodiment, and FIG. 4 illustrates a second embodiment of joint fixation devices, particularly usable on an animal such as a human. The devices 11 and 111 both include at least one magnetic member 12, and 112 respectively, and at least another magnetic device 12, 112 or another component made of a magnetic material. The devices 11 and 111 have magnetic components thereof positioned to either attract one another and/or repel one another. The devices 11 are shown in conjunction with their use on a spine; while the device 111 is shown in conjunction with its use with a ball and socket joint, e.g., a hip joint. The devices 11, 111 will be described in terms of both magnetic members 12, 112 in a device 11, 111 being magnets for simplicity. When the devices 11, 111 utilize magnets, the magnets will have a north or + and a south or − end. Like ends repel and unlike ends attract.

As used herein, the term “proximate end” defines the end closest to the user, i.e. patient, when in use.

As used herein, the “distal end” is defined as the end located farthest from the user when in use.

As used herein, “pedicle screw” or “pedicle screw assembly” is used to describe commonly used orthopedic or spinal surgical instrumentation, individually or as a unit, such as described in U.S. Pat. No. 7,066,937. The disclosure in this patent regarding the construction of a pedicle screw is incorporated herein by reference in its entirety. While many embodiments of a pedicle screw exist commercially, the typical pedicle screw assembly consists generally of the pedicle screw containing a threaded portion which is inserted into a bone or spinal vertebrae. Connected to the screw is a housing unit having upwardly shaped arms which form a U-shape unit. The housing unit is generally constructed to receive a longitudinal or spinal rod. The longitudinal or spinal rod is set to the housing through use of a set screw, which can be designed to screw into a threaded portion of the housing to lock the rod into place. This general construction scheme allows the surgeon to connect and secure adjacent bones together through use of the pedicle screw assembly, thereby providing stability temporarily until the bones heal or, if needed, permanently.

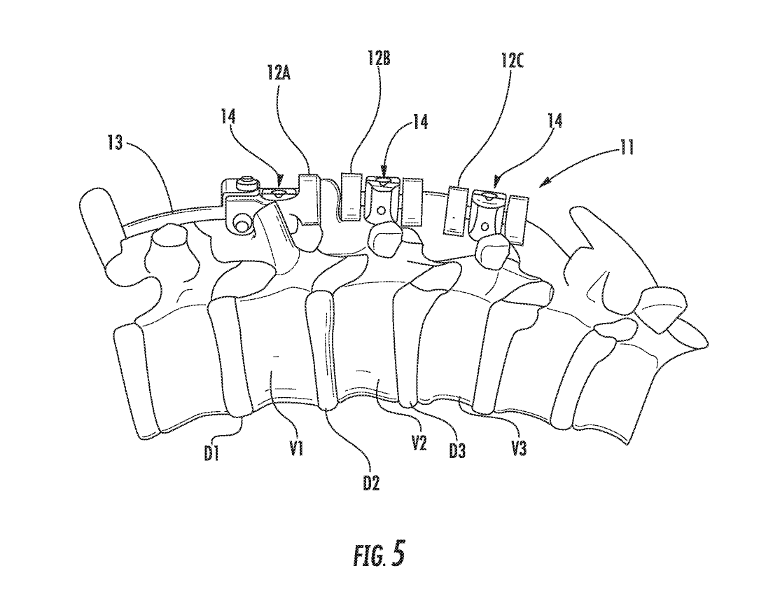

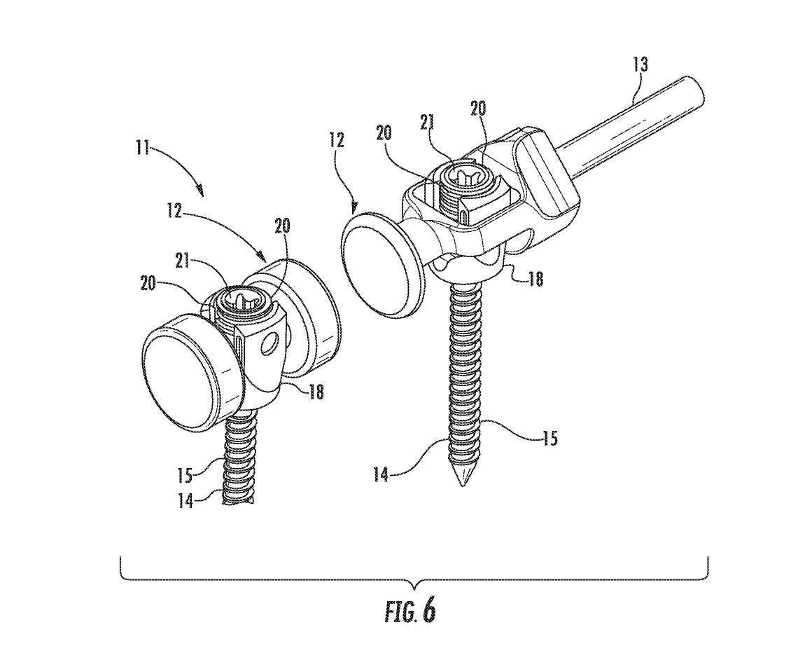

FIGS. 1A, 1B and 6 illustrate a pedicle screw 14 usable in the present invention as a portion of a fixation device 11. A spinal rod 13 can be secured to a pedicle screw 14, FIGS. 5, 6. The pedicle screw 14 includes a threaded shank 15 that is adapted for insertion into a bone such as a vertebra. The distal end of the threaded shank 15 has a portion of a ball 16, preferably integral therewith, and a tool socket 17 therein for the receipt of a fastening tool to install the threaded shank 15 into the bone with the screw 14, which also includes a polyaxial connector assembly 18 having a socket 19 receiving the ball 16 therein, which allows the longitudinal axes of the connector 18 and the screw 14 to be moved relative to one another. The connector 18 is also provided with a pair of opposed channel components 20, which can receive a portion of a magnet or magnetic member 12 for securement therein. The magnetic member 12 is secured in the connector 18 as with a set screw 21 threadably engaging an interior threaded surface of the connector 18.

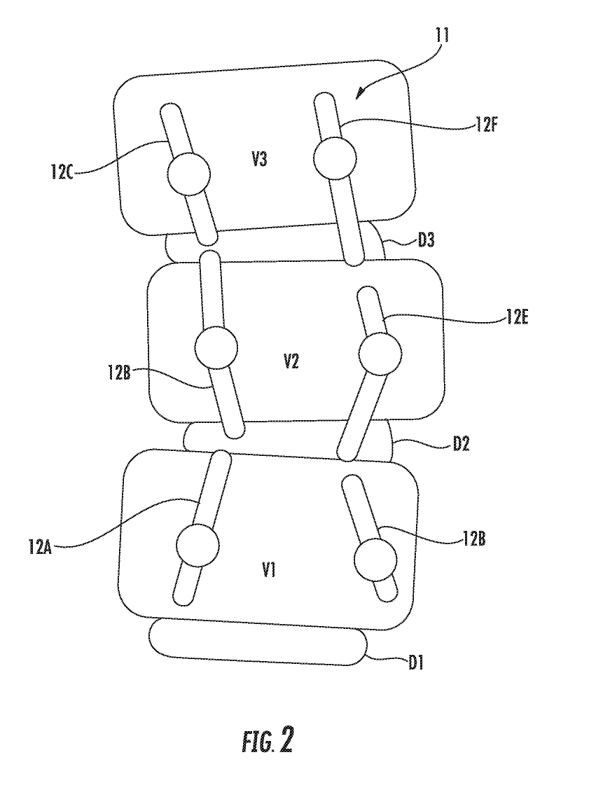

FIGS. 2, 5 illustrate, in schematic form, a portion of a spine. Three vertebra, designated V1, V2 and V3, and three intervertebral discs, designated D1, D2, D3, are indicated in joints between adjacent vertebrae. In the illustrated spine section, each of the adjacent vertebrae has a pair of magnetic members 12, such as a pair of magnets or a magnet and a magnetic material member, secured thereto with respective attachment devices such as pedicle screws 14. For clarity of discussion, the magnet members 12 are designated 12A-12F. In the illustrated embodiment of FIG. 2, each magnetic member 12A, 12B, 12C, 12D and 12F has a linear longitudinal axis. Magnetic member 12E has opposite end portions with longitudinal axes that are not linear to one another. In other words, the magnetic members 12 can have bends or curves in them if desired. Adjacent ends of adjacent magnetic members 12 can be spaced or touching as desired. Further, the magnetic members 12 may be encased in a polymeric material or the like to shield them from bodily fluid and direct body part contact. Further, as seen in FIG. 2, adjacent magnetic members, for example magnet members 12B and 12C, can have their respective longitudinal axis aligned with the longitudinal axis of the other. FIG. 5 shows the magnetic members 12A, 12B and 12C aligned. By contrast, the longitudinal axes of the magnetic members 12A and 12B are not aligned. Spacers can be provided to a surgeon to ensure proper spacing between adjacent magnetic member ends. An aligning tool can also be provided to a surgeon to properly fix the angle between adjacent magnetic members 12. A fixation device 11 includes at least one magnet 12 and its securement structure, such as the screw 14 and another magnetic member 12 in the securement structure. It is to be understood that one of the magnetic members 12 of an adjacent pair could be a magnetic component, i.e. one that is magnetically attractable to a magnet, but not a magnet. The magnetic member 12 can be rare earth magnets or other permanently magnetizable materials. N52 magnets are rare earth magnets that can currently produce the strongest magnetic fields in both repelling and attraction. The number indicates the magnetic field strength, while the letter N designates neodymium, a rare earth used in making magnets.

By aligning the longitudinal axes of adjacent magnetic members 12, a linear force can be applied between the two adjacent vertebrae across a joint. The force can either be attractive if opposite poles are adjacent one another, or repelling if like poles of two magnets are adjacent one another. If a magnet is adjacent a nonmagnet magnetic member, the force between is attractive. If the longitudinal axes of adjacent magnetic members 12 are set at angles relative to one another other than the linear, for example magnets 12A, 12B, either an attractive or repelling force can be applied to adjacent vertebra; and in addition, torque can be applied across a joint. This allows flexibility in how adjacent skeletal structures can be fixed relative to one another. An angle jig can be provided to the surgeon along with tables to establish, for given magnets 12, how much force and torque can be applied. While the above description contemplates adjacent magnets 12 both being magnets, it is to be understood that one of these components can also be made of a magnetic material as described above, and one made of a non-magnetizable material. Preferably, the magnets 12 are permanent magnets. While generally cylindrical magnets 12 are shown, their shape can be any suitable shape such as dumbbell shaped, generally spherical, rectangular solid, oval or the like as desired. In addition, the magnets may include one of several coatings which prevent the animal from rejecting, or the animal's body from attacking or resorbing, the magnetic material. Such coatings may include, but should not be limited to, polymeric coatings and the like.

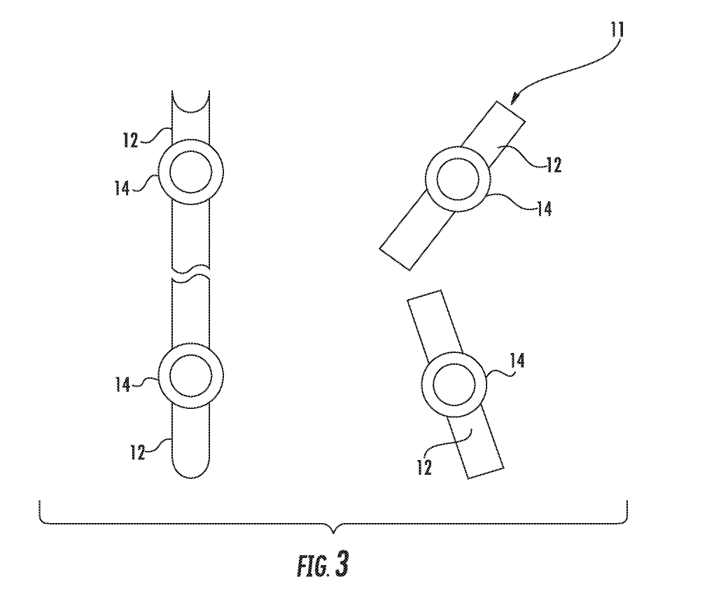

FIG. 3 illustrates variations of the present invention for the use of magnetic members 12. In the left-hand pair of magnetic members 12, the magnetic members 12 have linearly aligned longitudinal axes. It is also noted that the ends of these two magnetic members 12 are contoured. In the top magnetic member 12, the ends are concave; while in the bottom magnetic member 12, the ends are convex. This would be particularly advantageous if the magnetic members 12 are to touch one another in use, allowing both contact and angular misalignment. In the right-hand pair of magnetic members 12, the ends of both magnetic members 12 are planar. Also, this pair of magnetic members 12 has their longitudinal axes misaligned at an angle of approximately 45°, which would allow for the inducement of a torque to the adjacent skeletal components. The fixation devices 11 can also be used in combination with other devices, such as rods held in place with pedicle screws. In another embodiment, the magnetic members 12 and pedicle screws 14 may be positioned at the distal end of a typical spinal fusion construction, such as those used for scoliosis. These constructions utilize rods with the screws to fuse the spine. However, while these constructions straighten the spine and correct deformity, they often result in a condition referred to as adjacent facet syndrome failure. This causes arching of the spine adjacent to the fused construction. By placing the magnetic members 12 of the present system in the adjacent vertebrae, a constant force can be applied to the adjacent vertebrae to alleviate or prevent the arching often seen with this type of spinal fixation.

FIG. 4 illustrates another embodiment of the present invention. This figure illustrates a fixation device that can be used as a replacement joint, such as a hip joint, shoulder joint or elbow joint. These replacement joints are a ball in socket construction with bone structure on opposite sides of the joint. FIG. 4 illustrates a hip joint, while the fixation device for one type of joint would be similar for all joints and, for the sake of simplicity of discussion, only a hip joint will be discussed. The ball portion 50 of the joint is connected to the femur 51. The socket portion 54 of the replacement joint is connected to the pelvis 55. The construction and implanting of such replacement joints is well known in the art. The fixation device 111 includes portions of, or portions associated with, both the ball 50 and socket component 54. As described above, the fixation device 111 includes a pair of magnetic components including at least one magnetic member 112 and another magnetic component, which can be another magnet or a component comprised of a magnetic material as described above for the magnetic members 12. The fixation device 111 will be described in terms of it comprising two magnets 112. In the case of a ball and socket joint fixation device, the magnets 112 will be configured for an attracting force wherein opposite poles of the pair of magnets 112 will be adjacent one another to provide the attracting force. In the configuration of the fixation device 111 shown in FIG. 4, the ball 50 magnet 112 will have its positive pole positioned adjacent the negative pole of the magnet 112, which is a component part of the socket member 54. Preferably, the magnets 112 are each respectively embedded or encased in the respective ball 50 or socket member 54. It may be desirable to provide a low friction material positioned between the outer surface 56 of the ball 50 that engages the concave mating surface 57 of the socket member 54. The construction of the magnets 112 is such as to preclude or reduce applying a magnetically induced resisting rotational torque during rotation of the ball 50 in the socket member 54.

The above described joint fixation device 11, can be provided as a system in kit form. The kit can be provided pre-sterilized and packaged. The system includes a plurality of pedicle screws 14. It also includes a plurality of magnetic members 12, each adapted to be secured to a respective pedicle screw 14 as described above. At least one of the magnetic members 12 includes a permanent magnet. The system can also include instructions and warnings. The system can also include labeling information such as system contents and manufacturer information.

The above described joint fixation device 11 can be used in a method to fix relative skeletal bone positions across a joint. The method includes securing a first magnetic member 12 to a first skeletal bone, such as a vertebra V1. A second magnetic member 12 is secured to a second skeletal bone, such as a vertebra V2. The first and second skeletal bones having a joint therebetween, such as that occupied by a disc D2. At least one of the magnetic members 12 is a permanent magnet. Force is applied to said first and second skeletal bones through one of magnetic attraction and magnetic repelling between said first magnetic member and said second magnetic member. The applied force can be adjusted by adjusting the spacing between the magnetic members 12 and their angular position relative to one another. For attracting force, dissimilar magnetic pole ends of permanent magnets are positioned adjacent one another or one magnetic member 12 is a permanent magnet and the adjacent magnetic member 12 is not a permanent magnetic, i.e., made of a magnetic material. For repelling force, adjacent magnetic members 12 are both permanent magnets, and similar magnetic poles are positioned adjacent one another.

All patents and publications mentioned in this specification are indicative of the levels of those skilled in the art to which the invention pertains.

It is to be understood that while certain forms of the invention are illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A skeletal joint fixation device, the device including:
a first magnetic member secured to a first skeletal bone;
a second magnetic member secured to a second skeletal bone, said first and second bones being separated by a joint therebetween, said first and second magnetic members having portions positioned to apply force through one of magnetic attraction and magnetic repelling to said first and second skeletal bones across said joint, at least one of said first magnetic member and second magnetic member being a permanent magnet.

2. The joint fixation device of claim 1 wherein the first magnetic member being secured to a first vertebra and the second magnetic member being secured to a second vertebra of a human.

3. The joint fixation device of claim 2 wherein the first and second magnetic members being secured to said first and second vertebra with a respective pedicle screw.

4. The joint fixation device of claim 3 wherein both said magnetic members being permanent magnets.

5. The joint fixation device of claim 4 wherein said first and second magnetic members each having a north pole and a south pole and having opposite poles of said first and second magnetic members adjacent one another to provide an attractive force therebetween.

6. The joint fixation device of claim 4 wherein said first and second magnetic members each having a north pole and a south pole and having same poles of said first and second magnetic members adjacent one another to provide a repelling force therebetween.

7. The joint fixation device of claim 3 wherein said first and second magnetic members each having a longitudinal axis with said longitudinal axes being generally aligned.

8. A joint fixation device system in a kit, the system comprising:
a plurality of pedicle screws;
a first magnetic member adapted to be secured to a said pedicle screw;
a second magnetic member adapted to be secured to another said pedicle screw; and wherein
at least one of said first magnetic member and said second magnetic member including a permanent magnet.

9. A method of positionally fixing a plurality of skeletal bones having a joint therebetween relative to one another, said method including:
securing a first magnetic member to a first skeletal bone;
securing a second magnetic member to a second skeletal bone, said first and second skeletal bones having a joint therebetween;
applying force to said first and second skeletal bones through one of magnetic attraction and magnetic repelling between said first magnetic member and said second magnetic member, and wherein, at least one of said first magnetic member and said second magnetic member being a permanent magnet.
